(19) United States
(12) Patent Application Publication
(10) Pub. No.: US 2018/0051255 A1
(43) Pub. Date: Feb. 22, 2018

(54) THREE-DIMENSIONAL SCAFFOLD CULTURE SYSTEM OF FUNCTIONAL PANCREATIC ISLETS

(71) Applicants: Anson Joo L. ONG, San Antonio, TX (US); Jose OBERHOLZER, Chicago, IL (US); Xiao-Dong CHEN, San Antonio, TX (US); THE BOARD OF REGENTS OF THE UNIVERSITY OF TEXAS SYSTEM, Austin, TX (US)

(72) Inventors: Anson Joo L. ONG, San Antonio, TX (US); Jose OBERHOLZER, Chicago, IL (US); Xiao-Dong CHEN, San Antonio, TX (US)

(21) Appl. No.: 15/557,310

(22) PCT Filed: Mar. 24, 2016
(86) PCT No.: PCT/US16/23997

§ 371 Date: Sep. 11, 2017

Related U.S. Application Data
(60) Provisional application No. 62/138,231, filed on Mar. 25, 2015.

(51) Int. Cl. C12N 5/071; A61K 35/39

(52) U.S. Cl. CPC C12N 5/0677; A61K 35/39; C12N 2513/00; C12N 2506/1346; C12N 2533/52; C12N 2501/33; C12N 2533/90; C12N 2533/50

(57) ABSTRACT

A cell culture system including a silk fibroid scaffold, culture media, and pancreatic cells. The pancreatic cells grown in the tissue culture system have physiological and morphological features like those of in vivo pancreatic cells. The cell culture system can be used to produce a pancreatic tissue-specific extracellular matrix capable of inducing differentiation of pancreatic cell precursors into pancreatic cells.

### CROSS-REFERENCE TO RELATED APPLICATIONS

This application claims priority to U.S. Provisional Application No. 62/138,231, filed Mar. 25, 2015, which is incorporated by reference in its entirety.

### BACKGROUND OF THE INVENTION

### 1. Field of the Invention

The present invention relates generally to the field of cell biology. More particularly, it concerns production and use of cell culture systems for pancreatic islets and production and use of pancreatic islet-specific extracellular matrices for growth and differentiation of cells.

### 2. Description of Related Art

Diabetes is a major challenge for the national and global public health community in the twenty first century (American Diabetes Association, 2013). Complications of diabetes, such as cardiovascular disease, kidney failure, blindness and lower limb amputations, further extend the human and economic impact of this serious disease (American Diabetes Association, 2013). Although diabetes can be managed medically with different therapeutic regimens, current treatments neither cure the disease nor reverse its complications.

The replacement of a patient's insulin producing cells (β-cells) is a current advanced therapeutic option. Patients receiving allogeneic islet transplantation for type 1 diabetes have achieved insulin independence with normal blood glucose levels. However, by five years, only 10% of these patients remain insulin independent. Further, critical donor shortages, gradual loss of graft function over time, and the need for long-term immunosuppression to prevent immune rejection must be solved before this approach can become a viable standard therapy for type 1 diabetes (Barton, et al., 2012; Ryan, et al., 2005).

Therefore, development of strategies to preserve or regain secretory components in the pancreatic islets is essential for the management of patients with decreased insulin production. Development of these treatment strategies requires the establishment of a system capable of replicating the pancreatic islet “niche” to support the proliferation and differentiation of pancreatic islets.

Currently, the standard procedure for obtaining islets for transplantation involves enzyme digestion of donor pancreas tissue, purification of the islets using a Ficoll gradient, and culture on TCP less than 24 hours before infusion. It is known that one human pancreas contains about one million islets (Matsumoto et al., 2011). By these standard procedures, the average yield of islets is only 125,000 to 400,000 islets per pancreas (Matsumoto et al., 2011). For transplantation, ˜10,000 islet equivalents (IEQ)/kg body weight are required; thus, multiple transplants are necessary to achieve long-term insulin independence (Matsumoto et al., 2011). Therefore, as mentioned above, donor shortage is a major issue for this type of therapy.

Extracellular matrix (ECM) is an important component of the cellular niche in tissues, supplying critical biochemical and physical signals to initiate or sustain cellular functions (Chen, et al., 2008; Lai, et al., 2010). With advances in tissue engineering, the various scaffold biomaterials have been developed to mimic ECMs for tissue regeneration or repair (Nagaoka, et al., 2010). Among them, the materials that have been use to support the proliferation and differentiation of progenitor cells include chitosan, polyglycolic acid (PGA), poly-(l)-lactic acid (PLLA), poly (lactic-co-glycolic acid) (PLAG), poly(ethylene glycol)-terephthalate (PEFT/poly (butylene terephthalate (PBT) (Kagami, et al., 2008; Chan, et al., 2012; Chen, et al., 2005). However, these polymeric scaffolds can induce inflammation resulting from the acidity of their degradation products (Athanasiou, et al., 1996; Cancedda, et al., 2003). Another potential scaffold material, Matrigel, which contains basement membrane proteins secreted by EHS mouse sarcoma cells, has been used to grow primary epithelial cells in culture (Maria, et al., 2011). Although varying levels of success have been achieved with this product, it is not consistent with the long term goal to reconstitute the pancreatic islets niche (tissue-specific ECM) on a scaffold for controlling stem cell fate. Natural scaffold materials, especially silk, are desirable due to their wide ranges of elasticity (allowing tissue-specific scaffold formation), pore sizes (allowing tissue specific nutrition and oxygen access), low bacterial adherence, biodegradable, and low toxicity and immunogenicity (Leal-Egana & Scheibel, 2010). Recently, it has been reported that native extracellular matrix (ECM), generated by bone marrow (BM) cells, enhanced the attachment and proliferation of human and mouse bone marrow-derived mesenchymal stem cells (BM-MSCs) (Chen, et al., 2007; Lai, et al., 2010).

A tissue-specific ECM microenvironment is essential to provide chemical and physical cues to direct/govern multipotent stem cells in vivo and in vitro for tissue regeneration and repair (Chen, 2010; Costa, et al., 2012).

There remains a need for a tissue culture system to allow growth of pancreatic islets in such a way that they retain physiologically relevant features of pancreatic islets function. Also desirable are pancreatic tissue-specific three-dimensional (3D) scaffolds for pancreatic tissue engineering. In addition, it is desirable to obtain pancreatic islets-specific extracellular matrices to be used to differentiate pancreatic islets cell progenitors, including pluripotent stem cells, into pancreatic islets and to grow pancreatic tissue that can be used in a variety of therapies.

### SUMMARY OF THE INVENTION

Disclosed herein is a cell culture system comprising a silk fibroid scaffold (SFS), culture media, and pancreatic cells. In some embodiments, the silk fibroid scaffold is coated with fibronectin. The silk fibroid scaffold can also be depleted of any allergens or other substances harmful to mammals, including sericins, before being used in the cell culture systems or in the creation of the extracellular matrices of the present invention.

A variety of different pancreatic cell types can be used in the cell culture system. For example, in some embodiments the pancreatic cells comprise beta cells. In some embodiments, the pancreatic cells comprise islets. The pancreatic cells can also be primary pancreatic epithelial cells, and can be mammalian cells, including human or rat cells.

Advantageously, the inventors have discovered that human pancreatic cells grown on silk fibroid and bone marrow extracellular matrix scaffold produce a greater number of high quality cells. For example, in some embodiments, the pancreatic cells are arranged in three-dimensional cellular aggregates. In some embodiments, the pancreatic cells are globular in shape, in contrast to cells grown without SFS or extracellular matrix, which can be flat and round. In some embodiments, the pancreatic cells demonstrate a greater motility than those grown without SFS. In some embodiments, the pancreatic cells do not form a monolayer, in contrast to cells grown without silk fibroid scaffold. In some embodiments, the pancreatic cells, comprising human pancreatic islets, maintained on native ECM made by bone marrow stromal cells are capable of producing more insulin producing cells (β cells) than islets pre-maintained on tissue culture plastic.

The pancreatic cells grown on SFS retain other morphological features of functional pancreatic tissue. For example, in some embodiments, the pancreatic cells comprise granule structures. In some embodiments, the granule structures have an average diameter of approximately 0.3 μm, which is consistent with morphology of pancreatic cells in vivo. In some embodiments, the granule structures occupy more than half of the cytosol of the pancreatic cells. These granule structures are consistent with being pancreatic secretory granules. In some embodiments, the pancreatic cells express GLUT2 in the cellular membrane, another hallmark of functional pancreatic cells. In some embodiments, the granule structures and/or the pancreatic cells themselves are capable of secreting insulin.

As another indication that the pancreatic cells of the cell culture system of the present invention retain physiological functions of in vivo pancreatic cells, in some embodiments, the pancreatic cells are capable of secreting insulin in response to exposure to an insulin induction agonist, which can be glucose. In some embodiments, the pancreatic cells are capable of secreting insulin in response to exposure to glucose at a concentration of 28×10⁻³ M for 15 minutes in PBS solution. Secretion of insulin can be measured by any method known by those of ordinary skill in the art. In particular, insulin concentration can be monitored. In some embodiments, the pancreatic cells are capable of secreting an amount of insulin sufficient to increase the insulin concentration in the culture medium by at least a factor of 2 and/or at least a factor of 5 after exposure to 28×10⁻³ M glucose as compared to glucose activity in the culture medium before exposure to 28×10⁻³ M glucose. In some embodiments, the culture medium of the cell culture system comprises a insulin secretion agonist, including in some embodiments glucose. In some embodiments, the culture medium comprises insulin secreted from the pancreatic cells.

Another advantage of the present cell culture system is that in some embodiments the pancreatic cells retain the in vivo physiological property of being capable of constructing a three-dimensional extracellular matrix. This three-dimensional extracellular matrix is pancreas-specific in some embodiments, which makes it useful in maintaining the physiological function of in vitro cultures of pancreatic cells and in directing the differentiation of pancreatic cell progenitors, including pluripotent stem cells, into pancreatic cells. This can also be useful in generating pancreatic tissue, which can be used for therapy themselves or for testing of therapies in vitro. In other embodiments the three-dimensional extracellular matrix is an extracellular matrix generated from bone marrow cells. In one instance, the bone marrow cells used are stromal cells. In another instance, the extracellular matrix is synthesized using a three-dimensional silk fibroin scaffold.

The three-dimensional extracellular matrix of the present invention measures, in some embodiments, at least 5, 10, 15, 20, 25, 30, 35, 40, 45, 50, 60, 70, 80, 90, 100, 150, 200, 250, 300, 400, or 500 μm or more in each dimension, which has various advantages over matrices produced by cells that are not grown on silk fibroid scaffold. In some embodiments, the average height of the three-dimensional extracellular matrix measures between about 10 and 20 μm, 10 and 30 μm, 10 and 40 μm, 10 and 50 μm, 20 and 40 μm, 20 and 60 μm, 20 and 80 μm, 20 and 100 μm, 30 and 100 μm, 50 and 100 μm, 70 and 100 μm, 100 and 200 μm, 100 and 300 μm, 100 and 400 μm, 100 and 500 μm, or any range derivable therein. In some embodiments, the three-dimensional extracellular matrix comprises collagen type IV, which is a characteristic of matrices with in vivo physiological properties. As the pancreatic cells of some embodiments of the cell culture system are capable of producing a three-dimensional extracellular matrix, in some embodiments, the cell culture system comprises an extracellular matrix, which in some embodiments measures at least 5, 10, 15, 20, 25, 30, 35, 40, 45, 50, 60, 70, 80, 90, 100, 150, 200, 250, 300, 400, or 500 μm or more in each dimension. In some embodiments, the three-dimensional extracellular matrix comprises collagen type IV.

Also disclosed is a method of forming a pancreatic tissue-specific extracellular matrix comprising exposing the cell culture systems described above to ascorbic acid. A pancreatic tissue-specific extracellular matrix is an extracellular matrix with properties associated with the extracellular matrix found in the pancreas in vivo. In particular, a pancreatic tissue-specific extracellular matrix has the ability to support growth of pancreatic cells in such a way that the cells retain functional and morphological features of pancreatic cells in vivo. In some embodiments, a pancreatic tissue-specific extracellular matrix has the ability to induce, support, and/or help direct differentiation of pancreatic cell precursor cells to differentiate into pancreatic cells. In some embodiments, a pancreatic tissue-specific extracellular matrix has the ability to support growth of pancreatic tissue.

Ascorbic acid can be used to induce pancreatic cells to produce a pancreatic tissue-specific extracellular matrix. In some embodiments, the method includes a step of incubating the cell culture system for a time and under conditions sufficient for the pancreatic cells to achieve confluence. Confluence is defined as a property of a cell culture wherein the cells cover substantially all of the growth surface. In some embodiments, the pancreatic cells reach only partial confluence, which means that only a portion of the growth surface is covered by pancreatic cells. For example, in some embodiments, the pancreatic cells reach at least 80 percent confluence, at least 85 percent confluence, at least 90 percent confluence, at least 95 percent confluence, or at least 99 percent confluence. In some embodiments, exposing the pancreatic gland cells to ascorbic acid is performed after the pancreatic achieve confluence. In some embodiments, confluence is substantially complete (e.g. 100 percent coverage of the growth surface) before exposure to ascorbic acid. In some embodiments, the pancreatic cells reach only partial confluence (for example, 80%, 85%, 90%, 95%, or 99% coverage of the growth surface). In some embodiments, the pancreatic cells are exposed to ascorbic acid for eight days.

In some embodiments, the method of forming a pancreatic tissue-specific extracellular matrix further comprises decellularizing the extracellular matrix. Decellularizing means removing substantially all of the pancreatic cells. Decellularization is accomplished in some embodiments by incubating the pancreatic cells with a composition comprising Triton X-100 and NH₄OH. Also disclosed is the three-dimensional extracellular matrix produced by any of the methods described above.

Also disclosed is a three-dimensional extracellular matrix produced by pancreatic cells cultured on silk fibroid scaffold. In some embodiments, each dimension of the three-dimensional extracellular matrix measures at least 5, 10, 15, 20, 25, 30, 35, 40, 45, 50, 60, 70, 80, 90, 100, 150, 200, 250, 300, 400, or 500 μm or more. In some embodiments, the height of the three-dimensional extracellular matrix measures between about 100 and 200 μm, 150 and 250 μm, 200 and 300 μm, 250 and 350 μm, or 300 and 400 μm. In some embodiments, the extracellular matrix is essentially free of pancreatic cells. Pancreatic cells can be removed from the extracellular matrix by any method known to those of skill in the art. For example, the pancreatic cells can be removed by incubating with a composition comprising Triton X-100 and NH₄OH. In some embodiments, the silk fibroid scaffold is coated with fibronectin.

Also disclosed is a method of producing pancreatic cells, the method comprising incubating precursors of pancreatic cells with any of the three-dimensional extracellular matrices described above, including the three-dimensional bone marrow extracellular matrix. In some embodiments, the three-dimensional extracellular matrices of the present invention have the ability to support, induce, and/or direct the growth of pancreatic cells from pancreatic precursors. In some embodiments, incubating the precursors with the three-dimensional extracellular matrices can include plating the precursor cells on a surface comprising a three-dimensional extracellular matrix and maintaining growth and nutrient conditions sufficient to allow growth and/or differentiation. In some embodiments the three-dimensional extracellular matrix is made using cells from the same subject that the pancreatic cells are from. In some embodiments, the pancreatic cells are pluripotent stem cells, including in some embodiments, mesenchymal stem cells and/or cells derived from bone marrow and/or umbilical cord.

Also disclosed is a method of treating a pancreatic condition in a subject comprising providing to the subject the pancreatic cells produced by any of the methods described herein. The pancreatic cells can be provided to the subject in any way known by those of skill in the art, including, for example, implantation and/or injection.

Also disclosed is a method of differentiating cells comprising incubating cells with any of the three-dimensional extracellular matrices described above, including the three-dimensional bone marrow extracellular matrix.

Also disclosed is a method of producing pancreatic tissue comprising obtaining pancreatic cells or pancreatic precursor cells and incubating the pancreatic cells or pancreatic precursor cells with any of the three-dimensional extracellular matrices described above, including the three-dimensional bone marrow extracellular matrix. In some embodiments the three-dimensional extracellular matrix is made using cells from the same subject that the pancreatic cells are from. In some embodiments, the pancreatic precursor cells are pluripotent stem cells, including mesenchymal stem cells. In some embodiments, the pluripotent stem cells are derived from bone marrow or umbilical cord. Tissues produced by this method can be useful in a variety of ways. In some embodiments, there is disclosed a method of treating a pancreatic condition in a subject comprising providing to the subject the pancreatic tissue produced by any the methods described herein. Tissues produced according to the methods described herein can also be useful in testing potential therapeutics or in determining the biological function or result of a particular substance or condition.

Tissues produced in vitro yet retaining physiological features of in vivo tissues provide a particularly useful tool for monitoring the effects of proposed therapies or molecules on the physiological functions of the tissues. Accordingly, there is disclosed a method of testing the biological activity of a substance comprising obtaining any of the cell culture systems described above; adding the substance to the cell culture system; and measuring a parameter of the cell culture system to determine the effect of adding the substance to the cell culture system. Adding the substance to the cell culture system can comprise adding the substance to the culture medium. The culture medium can be exchanged for a culture medium comprising a particular substance or combination of substances to monitor the effects of the culture medium change on the physiological functions of the pancreatic cells. Measuring a parameter of the cell culture system can include, for example, observing growth rates or morphological features of cells. It can also include, for example, measuring the ability of the pancreatic cells to secrete insulin or other substances. Any biologically relevant parameter can be measured and monitored to determine the biological effect of exposing the cells to a substance or of changing any conditions of growth. Changes in the parameter being measured or monitored can be attributed to the presence of the substance or the change in growth conditions if a corresponding control does not show the same change. In some embodiments, the substance being tested is a candidate therapeutic to treat a condition, including, for example, disorders of the pancreas or an insulin related disease. In some embodiments, the condition is metabolic syndrome, prediabetes, diabetes, or a side effect of a medication or radiotherapy.

There is also disclosed a method of testing the biological activity of a substance comprising obtaining any of the extracellular matrixes described herein; incubating pancreatic cells or pancreatic precursor cells with the extracellular matrix; contacting the pancreatic cells or pancreatic precursor cells with the substance; and measuring an activity or property of the pancreatic cells or pancreatic precursor cells to determine the effect of contacting the pancreatic cells or pancreatic precursor cells with the substance. In some embodiments, the substance is a candidate therapeutic to treat a condition. In some embodiments, the condition is an disorder of the pancreas or an insulin related disease. In some embodiments, the substance is a cellular growth factor or cellular differentiation factor.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E-1A-B: BM-ECM enhanced human pancreatic islet adhesion (images provided by Dr. Oberholzer). (A) Islets incubated on TCP for 60 hrs. (B) Islets incubated on TCP coated with BM-ECM for 60 hrs. Note the presence of islets in (B). 1C-1E: Human pancreatic islets adhered to BM-ECM had more insulin producing β-cells (green) and less apoptotic cells (red) as shown using immunofluorescence staining (IF) (images provided by Dr. Oberholzer). (C) Islets collected after cultured on TCP; (D) BM-ECM adherent islets; (E) BM-ECM non-adherent islets.

FIG. 2—Illustrated preparation scheme of decellularized bone marrow stromal cell-derived ECM.

FIG. 3—Rat pSGECs cultured on SFS exhibited morphological and functional characteristics of salivary gland acinar cells. On the top row, representative micrographs of the morphology of pSGECs grown on TCP or SFS. On the bottom row, the left two figures show representations of histological staining of pSGECs grown on silk fibroin scaffolds (SFS). Rat submandibular (SM) and parotid (PG) gland epithelial cells cultured on SFS were sectioned and stained with hematoxylin and eosin (H&E), periodic acid-Schiff or (PAS). The graph on bottom row shows specific amylase activity of SM and PG cells grown on SFS or TCP. Mouse saliva was used as a positive control

FIG. 4—Illustrated overview of some embodiments of the disclosed approach.

FIG. 5—Structural characteristics of prepared SFS shown by scanning electron microscopy.

FIG. 6—Structural characteristics of BM stromal cells cultured on SFS shown by scanning electron microscopy.

FIG. 7—Characterization of cell-free BM-ECM. SEM image of cell-free BM-ECM. AFM image (60×60 μm) showed fibers that were discrete, linear, and highly-aligned; ECM depth ranged up to 320 nm. Two-photon microscopy revealed the native collagen architecture of the ECM (note that mixtures of purified/recombinant matrix proteins are undetectable using Two-photon microscopy). Other components were visualized by IF staining with specific antibodies against the indicated ECM proteins; nonspecific isotype IgG was used a negative control (not shown). Bar: 100 μm.

FIGS. 8A-8E—Rat (Lewis) islet preparation. (A) Freshly isolated islets, bar=200 μm; (B) Islet viability determined by AO (live islets stain green) and PI (dead islets stain red) and viewed using fluorescence microscopy, bar=200 μm; (C) freshly isolated islets cultured on TCP for 7 days (note islets form aggregates or are fused), bar=100 μm; (D) and (E) islets cultured on rat cell-free BM-ECM for 7 days, bar=200 μm, and 100 μm, respectively.

FIGS. 9A-9D—Rat (Lewis) islet morphology. Freshly isolated islets (Fresh) (A) are compared with islets cultured on TCP (B) or BM-ECM (C and D) for 2 weeks. Islets were removed from the culture surface, pelleted, fixed, and embedded in paraffin. Sections were cut (10 μm thick) and stained with H&E. Bar=200 μm.

FIG. 10—Immunofluorescent (IF) staining for insulin in freshly isolated rat (Lewis) islets (Fresh) or after culture on TCP or BM-ECM for 2 weeks. Paraffin sections were prepared as described in FIG. 9, and stained with an antibody against rat insulin (green fluorescence). Parallel sections were stained with non-specific isotype antibody as negative controls. Cell nuclei were stained with DAPI. Bar=200 μm. More fused islets were observed when cultured on TCP as compared to BM-ECM.

FIGS. 11A-11B—TEM images of insulin-containing secretory granules in rat (Lewis) islets cultured for 2 weeks on TCP (A) versus rat BM-ECM (B). Cultured islets were collected from the ECM or TCP, pelleted, fixed and prepared for TEM as previously described. Numerous β-granules can be seen in the cytoplasm, especially with islets cultured on BM-ECM. N: Cell nuclei. Bar=2 μm.

FIGS. 12A-12C—TEM images of the basement membrane of rat (Lewis) islets immediately after isolation (“Fresh”) (A) or after culture for 2 weeks on TCP (TCP) (B) or rat BM-ECM (BM-ECM) (C). Arrows indicate the basement membrane. Bars=2 μm for Fresh; and 500 nm for TCP and BM-ECM. N: Cell nuclei

FIG. 13—GSIS assay of islets cultured on the various substrates in low glucose (5.6 mM) for 60 mins followed by high glucose (16.7 mM) for a second 60 minutes. Insulin release into the media was measured and a Stimulation Index (SI) calculated. Total insulin levels in the islets after culture were also assayed and expressed as the mean±SD (n=3). *p<0.01, TCP vs. the other culture surfaces.

FIGS. 14A-14C—(A) MLIC assay. Vehicle: negative control; PHA (Phytohemaglutinin): positive control; and WF splenocytes (Sp): positive control. The data for the positive controls were significantly different vs. fresh Lewis or WF islets cultured on TCP and WF islets cultured on Lewis BM-ECM (p<0.05). (B) Induction of hyperglycemia in the Lewis rats after STZ dosing of 80 mg/kg and STZ-induced hyperglycemia in Lewis rats was reversed by a single transplantation of freshly isolated Lewis islets through hepatic portal vein infusion. (C) Islet infusion into the portal vein, during survival surgery, is shown. (portal vein shown at the arrow) L: Liver

### DESCRIPTION OF ILLUSTRATIVE EMBODIMENTS

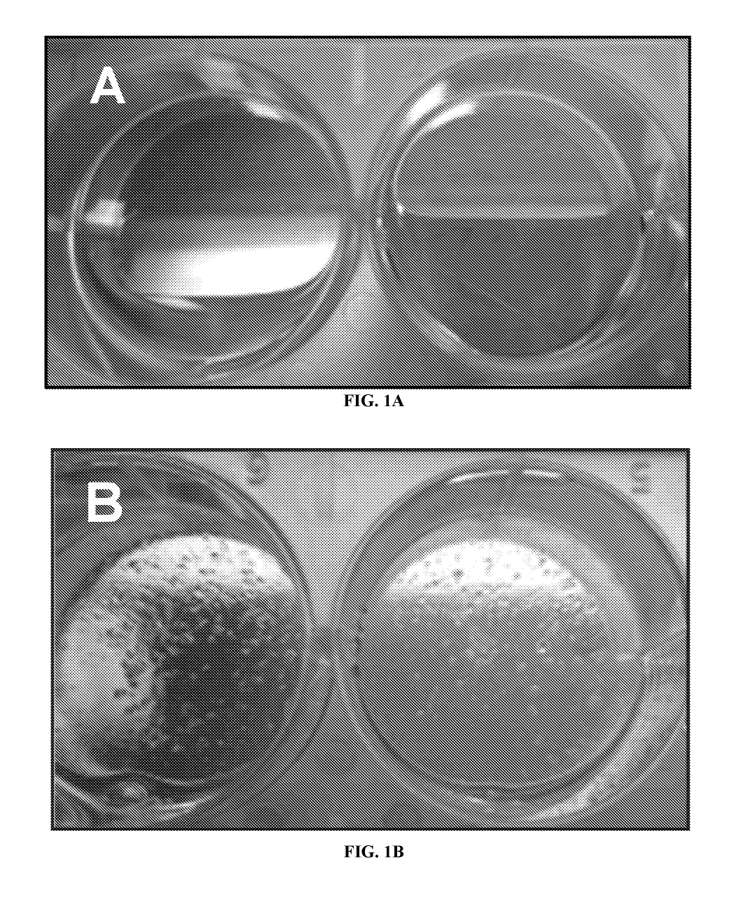

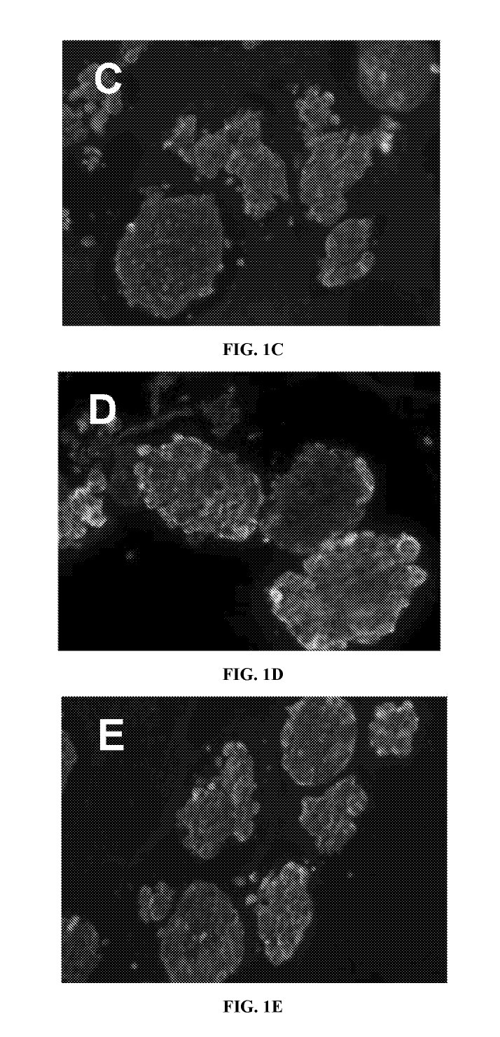

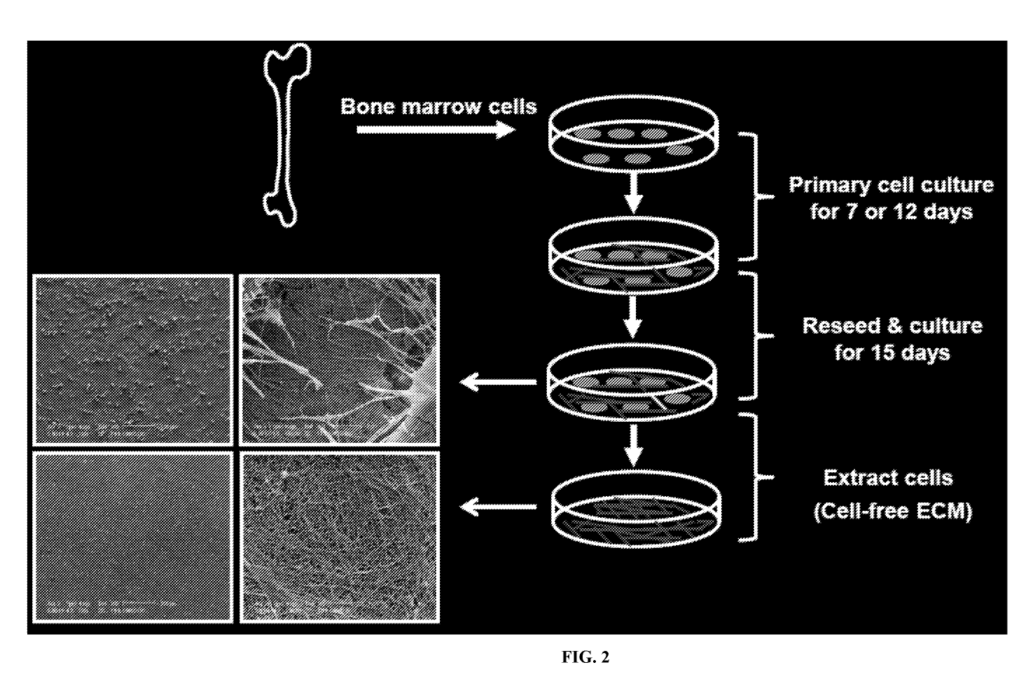

The present inventors examined the behavior of human pancreatic islets cultured with a unique native extracellular matrix (ECM) made by bone marrow (BM) (BM-ECM) to retrieve a larger number of high quality, insulin producing, human pancreatic islets than possible culturing with standard tissue culture plastic (TCP) (FIGS. 1B and 1A, respectively). The inventors disclose herein that human pancreatic islets pre-maintained on native ECM made by bone marrow stromal cells contained more insulin producing cells (β cells) than islets pre-maintained on TCP.

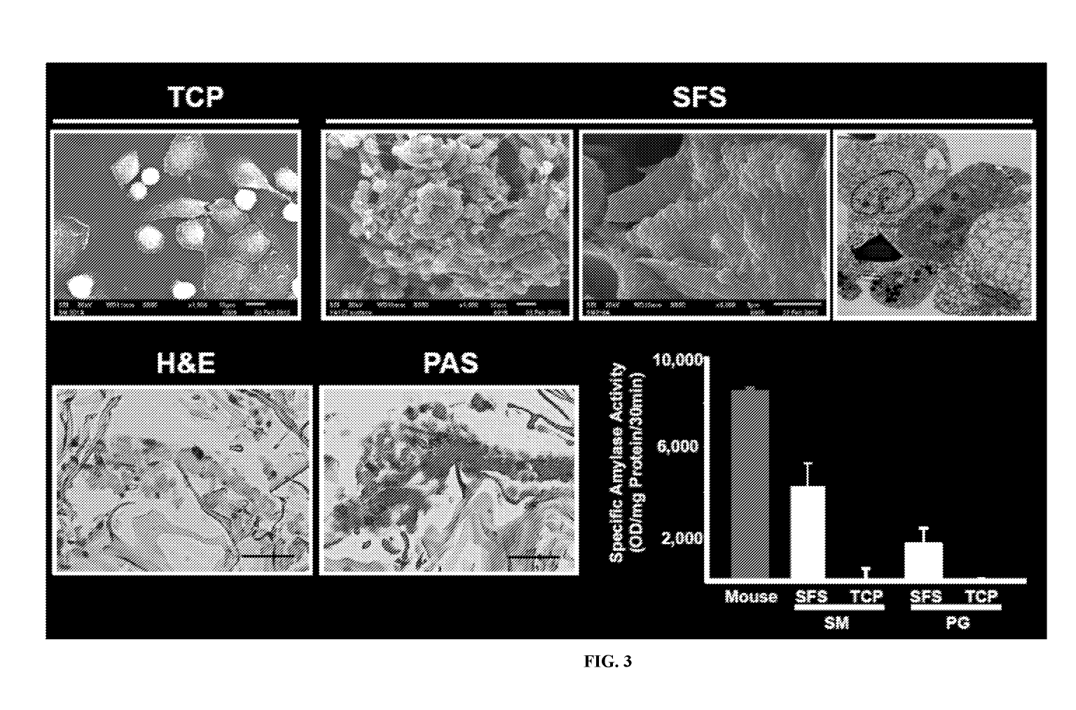

Recently, the inventors developed an authentic tissue-specific microenvironment (niche) ex vivo using three dimensional silk fibroin scaffolds (SFS) “coated” with tissue-specific ECM. This approach demonstrated that primary salivary gland epithelial cells (pSGECs) grown on SFS, but not tissue culture plastic (TCP), retain functional and structural features of differentiated salivary glands and produce an ECM that mimics the native salivary gland cell niche (PCT/US2015/014994, which is incorporated herein in its entirety by reference), see also FIG. 3. These unexpected, novel findings suggest that SFS provides a unique three-dimensional environment which allows cells to faithfully recapitulate their original phenotype in culture.

Both pancreatic islets and salivary gland are of epithelial origin; thus, this approach, using ECM-coated SFS, is expected to provide a culture system capable of producing an enriched population of high quality pancreatic islets with preserved differentiated function. Further, the risk of immune rejection is expected to be attenuated by “re-educating” the cells prior to transplantation by pre-exposure to BM ECM synthesized by cells of the recipient. The immunogenicity of allogeneic cells is expected to be attenuated by pre-exposure of the cells to the recipient's (host) environment. This approach overcomes two major issues, donor shortage and the need for life-long immunosuppression.

The studies described herein indicate that human pancreatic islets attached to BM-ECM contain a greater number of healthy β-cells, determined by stronger positive staining for insulin, and fewer apoptotic cells as compared to islets not attached to the BM-ECM or islets cultured on TCP (FIGS. 1D, 1E, and 1C respectively). This advanced technology is useful for reliably obtaining large numbers of high quality, low immunogenicity pancreatic islets. This technology is also expected to remarkably improve clinical outcomes.

Disclosed herein is a unique three-dimensional culture system for preparing therapeutically significant numbers of pancreatic islet cells for transplantation. Furthermore, attenuation of the immunogenicity of allogeneic transplant islet cells is expected to be achieved by pre-exposing them to an ECM generated by cells from the recipient. This culture system is further expected to mimic the islet in vivo microenvironment, which results in enhanced islet attachment, growth, and differentiated function.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

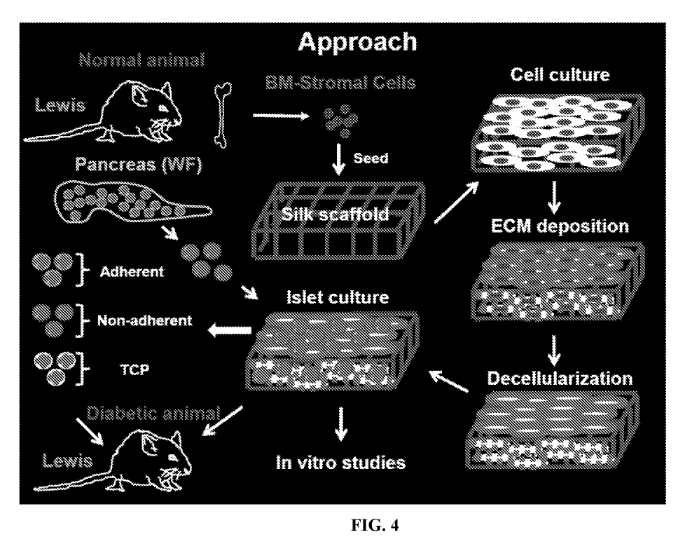

FIG. 4 illustrates an embodiment of the general approach used for the Examples below.

### Example 1 Culturing Human Pancreatic Islets on ECM-BM

Recently, it has been reported that native extracellular matrix (ECM), generated by bone marrow (BM) cells (BM-ECM), enhanced the attachment and proliferation of human and mouse bone marrow-derived mesenchymal stem cells (BM-MSCs) (Chen, et al., 2007; Lai, et al., 2010). Herein the inventors disclose that using BM-ECM to culture human pancreatic islets allow one to retrieve a larger number of high quality, insulin producing, human pancreatic islets than possible using tissue culture plastics (TCP) (FIG. 1).

Methods:

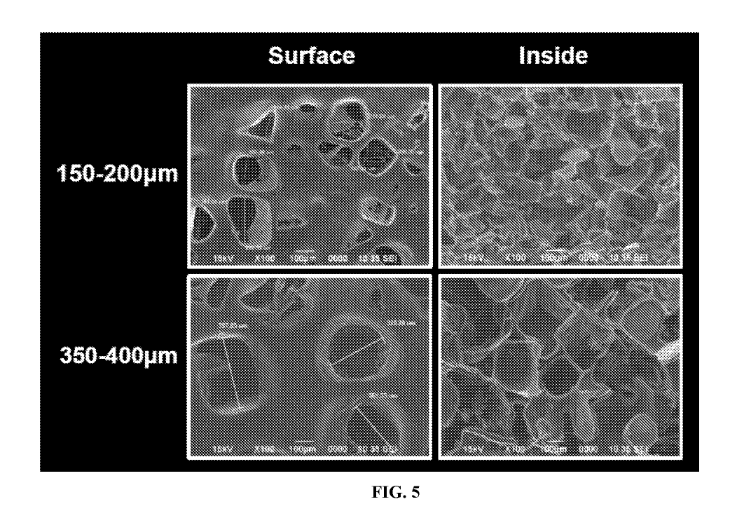

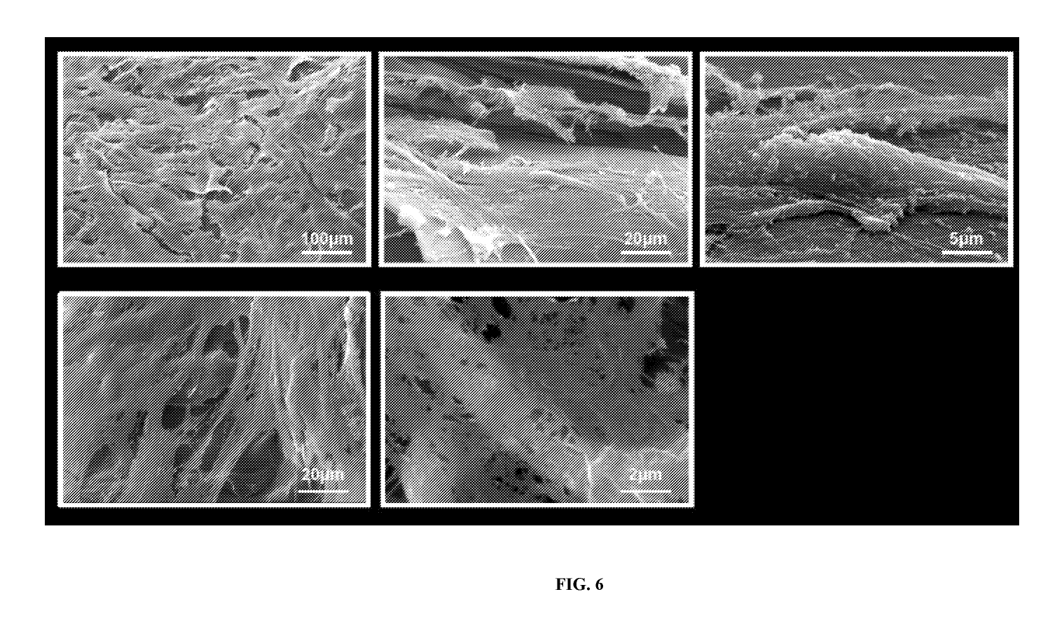

Native extracellular matrix (ECM), generated by bone marrow (BM) cells, was prepared as described below in Example 2 and in Chen, X. D, et. al. 2007, and Lai, Y, et al. 2010. FIG. 2 illustrates a general overview of the procedure. FIG. 5 is scanning electron microscopy figures of the structure of the SFS as prepared by the procedures. FIG. 6 is scanning electron microscopy figures of the structure of the BM stromal cells cultured on SFS as prepared by the procedures.

Using standard tissue culture procedures, freshly isolated human islets were seeded directly onto TCP or TCP coated with human BM-ECM at 200 islet equivalents (IEQ)/cm² and incubated for 60 hours.

Non-adherent and adherent islets were counted and stained with antibody to insulin (green) as well as transferase-mediated dUTP nick-end labeling (TUNEL) to identify apoptotic cells (red).

Results:

A larger number of islets were produced when incubated on TCP coated with BM-ECM (FIG. 1B) than on TCP (FIGS. 1B and 1A respectively). 60% of the total islets cultured on BM-ECM adhered to the BM-ECM while far fewer adhered to TCP.

The human pancreatic islets adhered to BM-ECM had more insulin producing β-cells and less apoptotic cells than the islets cultured on TCP or islets that did not adhered to BM-ECM (FIGS. 1D, 1C, and 1E, respectively).

### Example 2 Synthesis and Characterization of BM-ECM on TCP

A tissue-specific three-dimensional environment was developed using SFS, with varying degrees of porosity and interconnectivity, and “coated” with native BM stromal cell-derived ECM.

Synthesis of BM-ECM on TCP:

SFS is prepared using a previously described technique (Nazarov, et al., 2004; Sofia, et al., 2011). Briefly, Bombyx mori cocoons were purchased from Paradise Fibers (Spokane, Wash.) and processed to remove sericin from the silk fibroin. The silk fibers were dissolved in 9.5M LiBr, dialyzed vs. water, and lyophilized. The samples were then rehydrated, sonicated, poured into Teflon molds, and lyophilized to create thin films. The protein structure of the resulting silk film were converted from α-helix to β-sheet by treatment with methanol, followed by washing and sterilization before use. A salt leaching process was used, after the last lyophilization step, to produce scaffolds of varying pore sizes and interconnectivities; NaCl crystals of 3 different size ranges (100-200, 200-300, and 300-400 μm) and different weight ratios of NaCl to silk (10:1, 15:1, and 20:1) resulted in 10 different scaffolds, including the unmodified SFS. The selected pore sizes are based on an average islet size (islet equivalent [IE]) of 150 μm in diameter (Scharp, et al. 2014; Daoud, et al., 2010) with sizes ranging from 75-400 μm (Scharp, et al. 2014).

BM-ECM can be synthesized on SFS (ECM-SFS) according to a previously published method (Lai, et al., 2010). Briefly, rat bone marrow stromal cells (passage 2) were reseeded onto the SFS and cultured for 15 days; ascorbic acid (50 μM) was added to the media during the final 8 days of culture. At harvest, the stromal cells on the SFS were removed using a decellularization procedure as described previously (Chen, et al., 2007; Lai, et al., 2010).

Characterization:

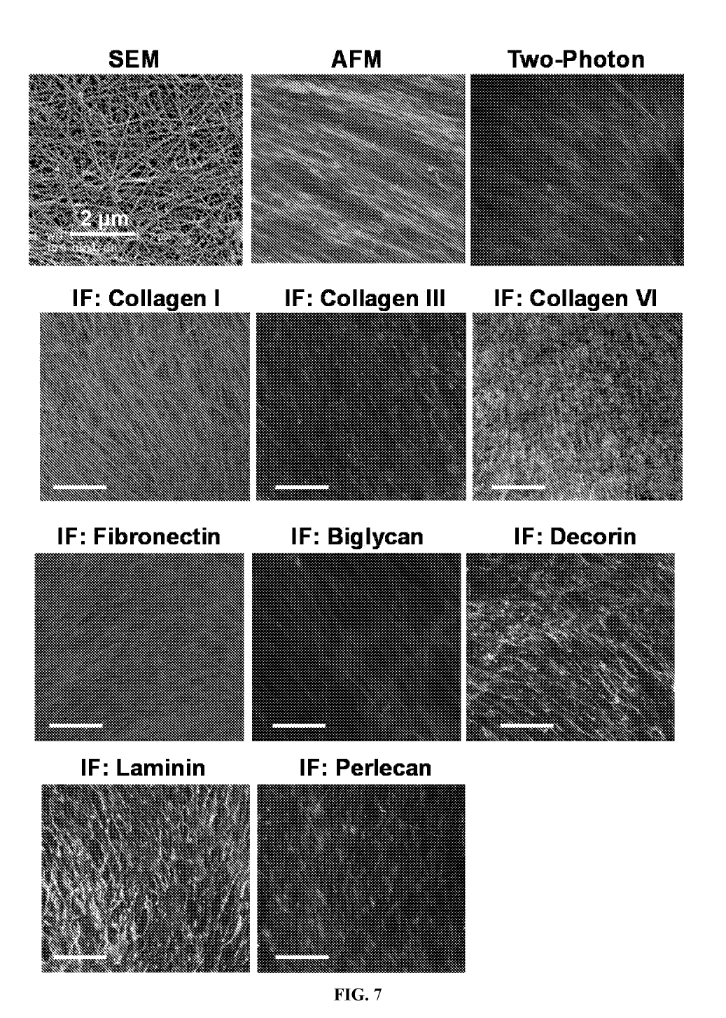

Scanning electron microscopy (SEM) was used to capture high resolution digital images (JEOL 7500) for the evaluation of the BM-ECM on TCP. The BM-ECM on TCP displayed a well-organized structure (FIG. 7). Further evaluation of this ECM, using atomic force microscopy (AFM), and second-harmonic imaging microscopy (SHIM; two-photon) (FIG. 7), revealed the architecture of the collagenous matrix. By mass spectrometric analysis, over 140 different proteins were identified and collagen VI was the most abundant. Coincidentally, adult pancreas has been reported to be especially enriched in collagen VI. The presence of a number of proteins that are known to be important for maintenance of islets were confirmed to be present in the BM-ECM on TCP by use of immunofluorescence staining (IF) (FIG. 7). The proteins include collagen I, collagen III, collagen VI, fibronectin, biglycan, decorin, laminin, and perlecan.

Pore size, interconnectivity and morphology of the SFS can also be determined. Porosity can be calculated using helium pycnometry (AccuPyc 1340) to measure scaffold volume and a Micromeritics ASAP 2020 can be used to calculate surface area per mass (cm2/g) utilizing Brunauer-Emmett-Teller (BET) theory. The pycnometer and BET values can then be used to calculate the surface to volume ratio. An atomic force microscope (Veeco Multi-Mode V Scanning Probe) can be employed to determine the morphology and mechanical properties of the scaffolds (Wang, et al., 2004) Target values for scaffold stiffness are based on the fact that pancreatic tissue has a rigidity of around 3.1 kPa and INS-1E cells (β cell line) have been shown to display augmented growth and attachment with substrate rigidities between 1.7-64.8 kPa (Naujok, et al., 2014) Further, enhanced response to glucose stimulation has been demonstrated with values of 0.1-10 kPa (Nyitray, et al, 2014). Using the described design and targets for scaffold characteristics, the optimal combination of scaffold properties in Example 3 can be determined.

### Example 3 Characterization and Comparison of Rat Pancreatic Islets Cultured on BM-ECM or TCP

The efficacy of the ECM-SFS culture system in promoting pancreatic islet attachment, growth, and differentiated function was determined by culturing rat pancreatic islets on rat or human BM-ECM and compared to those cultured on TCP. BM-ECM with varying pore size and interconnectivity can also be compared.

Preparation of Rat Pancreatic Islets:

Inbred Lewis or Wistar-Furth (WF) rats (250-300 g) were purchased from Harlan (Dublin, Va.) and used to obtain islets for allograft and isograft. Pancreatic islets were harvested using collagenase XI (1 mg/ml) (Roche, Ind.) perfusion through the common bile duct and purified by continuous-density Ficoll gradient (Carter, et al., 2009). 500 to 700 islets/pancreas with ˜90% purity (FIG. 8A) were isolated. Viability of the purified islets was about 85% using Acridine orange (AO)/propidium iodide (PI) staining (live islets stain green with AO; dead islets stain red with PI) (FIG. 8B).

Culture of Rat Pancreatic Islets:

Varying amounts of islets (e.g. 200, 600, and 2000 IEQ/cm3) were load onto TCP and rat BM-ECM scaffolds (prepared in Example 2) and cultures for multiple days.

Structural Characteristics of Cultured Islets:

Freshly isolated islets cultured on TCP for 7 days formed aggregates and did not adhere well (FIG. 8C). In contrast, freshly isolated islets cultured on rat BM-ECM for 7 days were evenly distributed and did not aggregate. Interestingly, islets not only adhered better to the ECM, but more fibroblast-like cells grew out from around the islets (FIGS. 8D and 8E). Moreover, the surface of individual islets appeared smoother and more uniform after culture on the BM-ECM compared to TCP. This suggests that “passenger” cells migrated out from the islets during culture on the BM-ECM and may carry fewer contaminating cells than islets cultured on TCP.

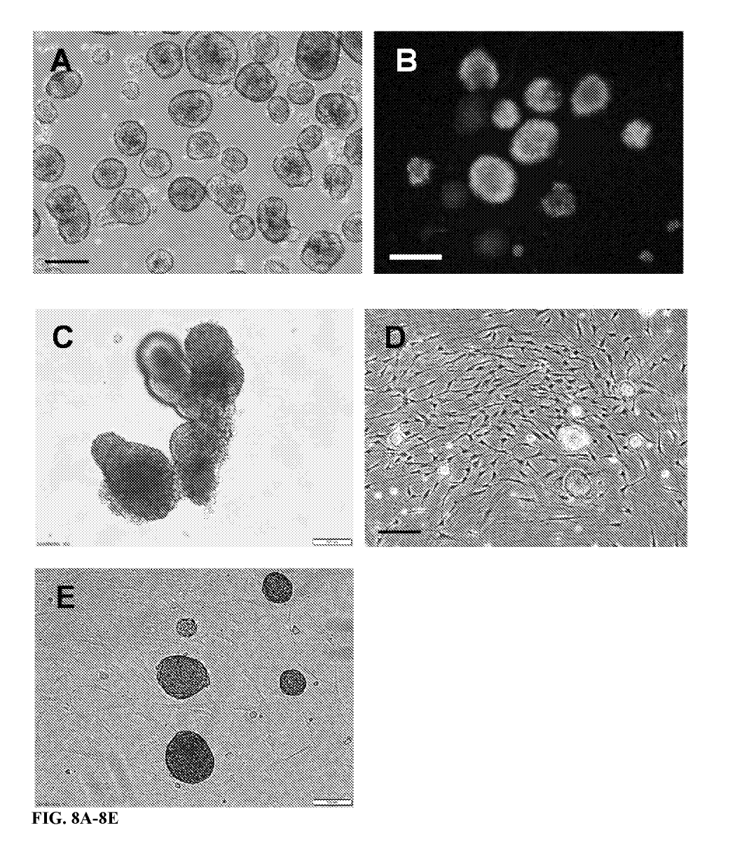

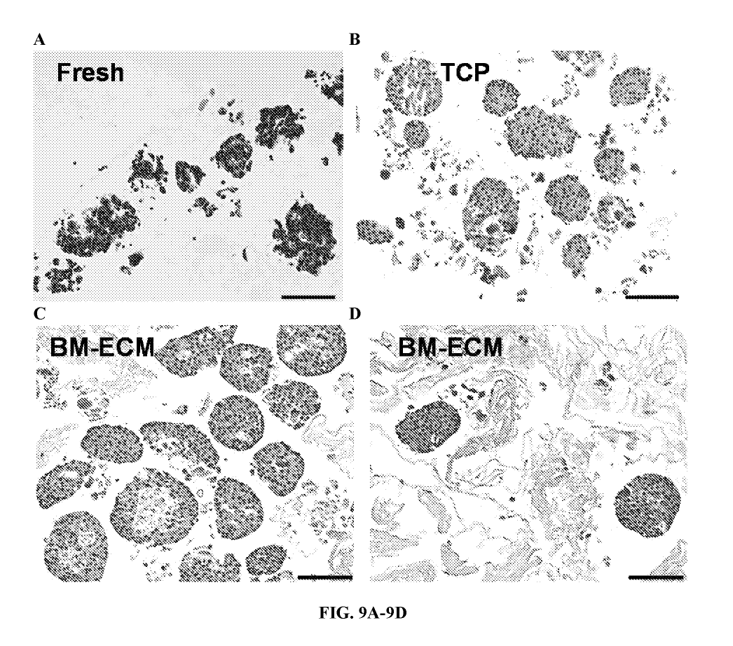

Rat islets cultured on BM-ECM were larger in size and had a smooth surface compared to freshly isolated islets or after culture on TCP. Freshly isolated rat islets were relatively small and had a rough surface (FIG. 9A). After culture for 2 weeks on rat BM-ECM, not TCP, rat islets appeared larger in size and had a smoother surface; some islets retained intimate contact with the surrounding matrix (FIGS. 9C and 9D), suggesting a better recovery from damage caused by isolation, but this was not found on TCP (FIG. 9B).

Insulin Production of Cultured Islets:

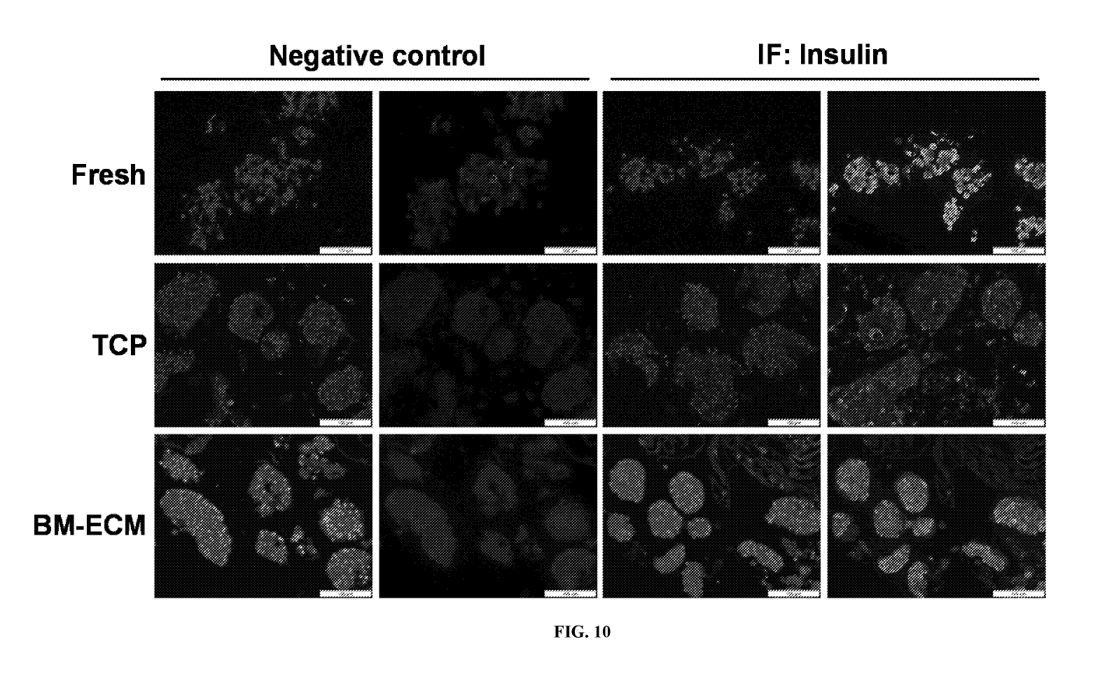

It was demonstrated that rat islets produce more insulin with culture on BM-ECM than TCP. Briefly, islets that were freshly isolated, or cultured on BM-ECM or TCP for 2 weeks, were stained with rat insulin antibody and observed in the fluorescent microscope at the same exposure setting (FIG. 10). Islets cultured on BM-ECM exhibited brighter IF staining than those cultured on TCP (FIG. 10). Freshly isolated rat islets served as a positive control

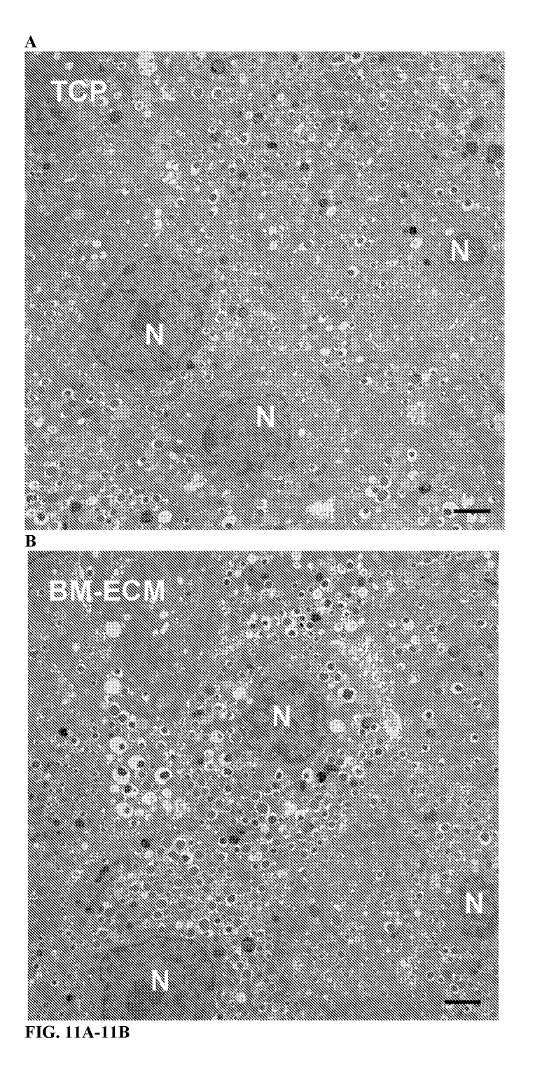

Consistent with the IF results shown in FIG. 10, transmission electron microscopy (TEM) showed that β-cells in islets cultured on rat BM-ECM for 2 weeks had both greater numbers and larger size insulin-containing secretory granules than islets cultured on TCP (FIGS. 11A and 11B). Together, these results (FIGS. 10, 11A, and 11B) provide strong evidence that islets cultured on BM-ECM contain higher levels of insulin compared to TCP.

Islet Basement Membrane Integrity of Cultured Islets:

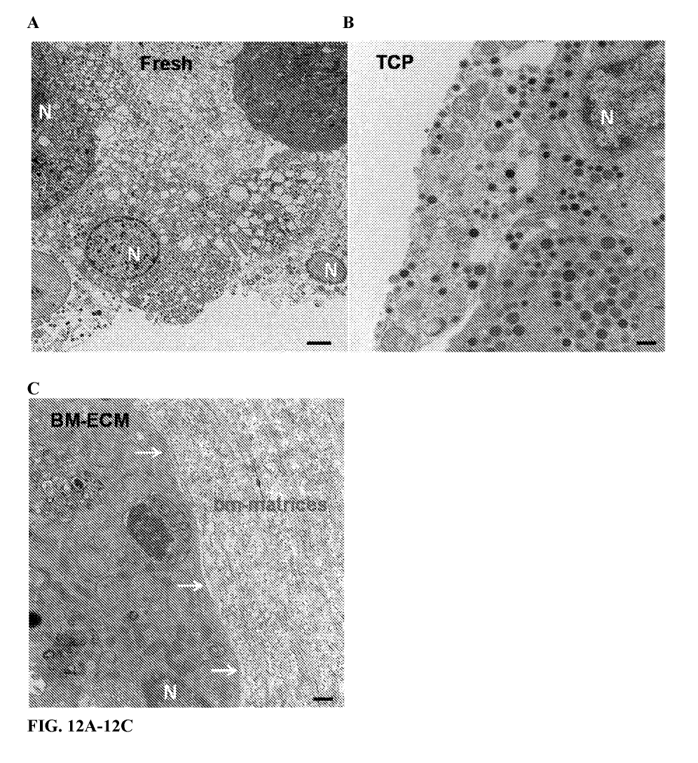

Rat islet basement membrane integrity is restored with culture on rat BM-ECM. TEM showed the complete absence of a basement membrane in freshly isolated islets and only a partial (incomplete) basement membrane after culture on TCP for 2 weeks (FIGS. 12A and 12 B). These “naked” or severely damaged islets may also be contaminated with unknown amounts/various types of “passenger” cells such as macrophages or other MEW class II antigen presenting cells. In contrast, islets cultured on BM-ECM for 2 weeks formed a tight boundary with the bone marrow matrix (bm-matrices) clearly containing collagen fibrils (FIG. 12C). The basement membrane that formed at this junction was very smooth. Remarkably, culture on BM-ECM promoted the restoration of the islet basement membrane and may partially explain the results seen in FIGS. 9A, 9B, 9C, 9D, and 10.

Insulin Production in Response to Glucose Stimulation on BM-ECM Produced from Rat and Human Donors:

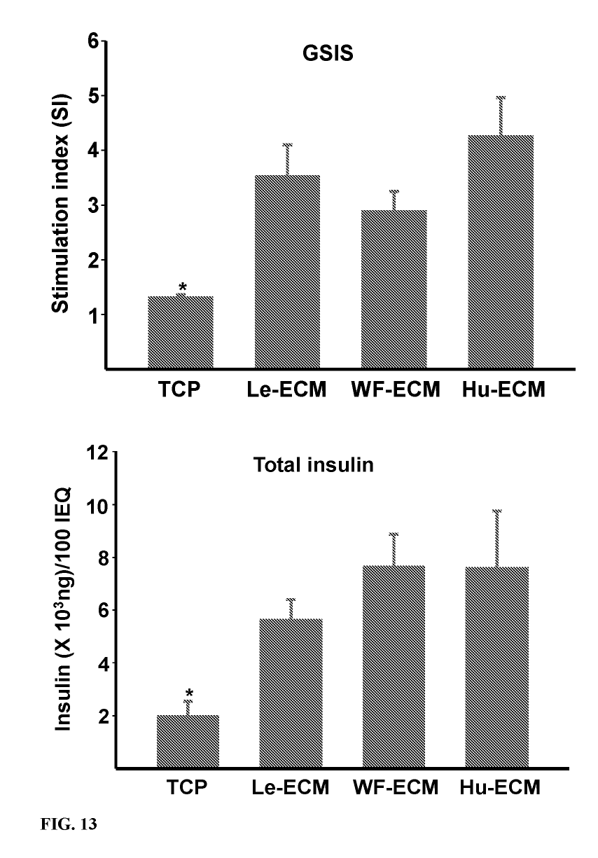

Rat islets cultured on BM-ECM produce greater quantities of insulin in response to glucose stimulation than on TCP. Briefly, to assess the functional capacity of islets cultured on the various substrates, a glucose-stimulated insulin secretion (GSIS) assay was performed (FIG. 13). Rat (Lewis) islets were cultured for 2 weeks on TCP or BM-ECM produced by BM stromal cells from rat (Lewis [Le-ECM] or Wistar-Furth [WF-ECM] or human (Hu-ECM) donors. For the assay, the islets were pre-incubated with “low” glucose (5.6 mM) Krebs-Ringer buffer for 60 minutes and then switched to “high” glucose (16.7 mM) in Krebs-Ringer buffer for a second 60 minute incubation. Rat insulin levels in the media were measured using a rat insulin ELISA kit (Wako Chemicals, USA) and a stimulation index (SI) calculated by dividing the mean insulin values (normalized to DNA content) measured in the high glucose treated cultures by that measured in the low glucose cultures. FIG. 13 shows that the islets maintained on BM-ECM, irrespective of strain or species, produce a significantly higher amount of insulin in response to glucose stimulation. In addition, the total amount of insulin contained in the islets cultured on the BM-ECMs was also higher than on TCP.

Rat Pancreatic Islet Immunogenicity:

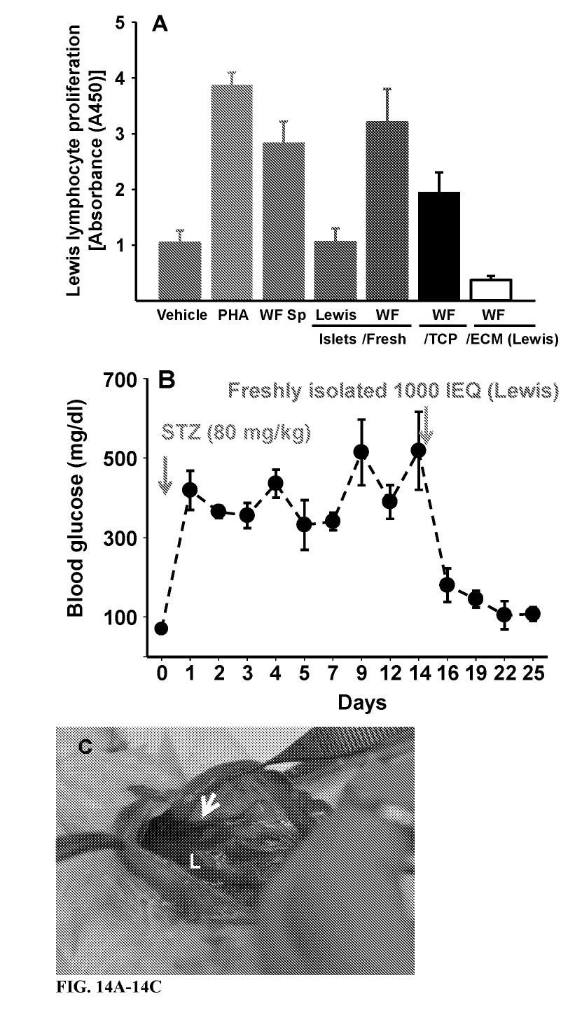

Pre-culture on rat BM-ECM attenuates rat pancreatic islet immunogenicity. The effect of culture on BM-ECM on the immunogenicity of allogeneic islets was determined using a mixed lymphocyte islet culture (MLIC) assay. Briefly, WF islets were pre-cultured on either TCP (FIG. 8C) or BM-ECM, made by Lewis rat bone marrow cells, for 7 days (FIG. 8D). Then, islets were treated with mitomycin C for 30 minutes to suppress proliferation, followed by co-culture with Lewis rat splenocytes containing T lymphocytes. Sixteen hours prior to harvest, BrdU was added to the media, and cell proliferation was measured using a cell proliferation ELISA kit. FIG. 14A shows that WF islets, pre-cultured on Lewis rat ECM, failed to stimulate Lewis lymphocyte proliferation. This response was in contrast to freshly isolated WF islets and WF islets pre-cultured on TCP that both elicited a strong proliferative response from the Lewis lymphocytes. More interestingly, the reaction to the WF islets cultured on Lewis BM-ECM was even lower than that observed with isogeneic islets (Lewis islets to Lewis lymphocytes).

Additional Assays:

Additional assays known in the art can be used to characterize islets. The optimal dose of islets and combination of scaffold porosity and interconnectivity which maximizes the attachment, growth, and differentiated function of islets can be identified. Culture on ECM-SFS is expected to attenuate the immunogenicity of the islets in the in vivo assay in Example 4. ECM-SFS is expected to significantly increase the surface area for carrying more islets than ECM alone. Optimal porosity and interconnectivity can be identified based on the combination which yields the highest number of islets of high quality (i.e., differentiated function). Islet immunogenicity can be determined by in vivo functional assay of the transplanted islets (see Example 4). ECM synthesized by pancreatic fibroblasts on SFS can also be used to retain islet function.

### Example 4 Reversal of Streptozotocin (STZ)-Induced Hyperglycemia

Transplantation of freshly isolated islets, via hepatic portal vein infusion, reverses streptozotocin (STZ)-induced hyperglycemia. It is expected that islets obtained using the ECM-SFS culture system will demonstrate anti-diabetic properties in a streptozotocin (STZ)-induced diabetic rat model and in diabetic subjects, including humans.

Rat Model of DM1 and Reversal by Transplantation of Freshly Isolated Islets—

A rat model of DM1 has been established and described herein using a single injection of STZ (80 mg/kg i.p.) (FIG. 14B). Briefly, inbred and outbred male Lewis rats (250-300 g) were purchased from Harlan (Dublin, Va.) and diabetes (type 1) was induced via intravenous injection of STZ (King, 2012). FIG. 14B shows the induction of hyperglycemia in the Lewis rats after STZ dosing. Hyperglycemia in these animals was induced in approximately 1-2 days and was maintained for more than 8 weeks. These animals were been successfully treated by transplantation of 1,000 isogenic islets via hepatic portal vein infusion (FIGS. 14B and 14C).

Reversal of DM1 by Transplantation of BM-ECM Cultured Islets:

Isogenic (between inbred) and allogeneic (between outbred) islets (2000 IE/kg), obtained using the ECM-SFS constructs identified in Example 3, can be transplanted through hepatic portal vein infusion (n=6 per group) as performed in patients; negative controls can receive saline. Body weight and blood glucose levels can be measured at the same time of day starting the day before transplantation and at weekly intervals thereafter. Plasma insulin can be measured using a rat C-peptide ELISA Kit (Crystal Chem Inc, IL). Ninety days after transplantation, a glucose tolerance test can be performed immediately before necropsy. At necropsy, liver tissue can be harvested for measurement of islet size and beta-cell mass (Do, et al, 2012).

The optimal dose of islets and combination of scaffold porosity and interconnectivity which maximizes the function of islets in vivo can be determined by the method above. Islet immunogenicity can be determined by in vivo functional assay of the transplanted islets.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
American Diabetes Association 2013. Economic Costs of Diabetes in the U.S. in 2012. Diabetes Care, 2013.
Athanasiou, et al., Biomaterials. 17(2):93-102, 1996.
Barton, et al., Diabetes Care. 35:1436-1445, 2012.
Cancedda, et al., Matrix Biol. 22(1):81-91, 2003.
Carter, et al., Biol. Proced. Online. 11:3-31, 2009.
Chan, et al., Biomaterials. 33(2):464-72, 2012.
Chen, et al., J. Bone Miner. Res. 22:1943-1956, 2007.
Chen, et al., Tissue Eng. 11(3-4):526-34, 2005.
Daoud, et al., Biomaterials. 31:1676-1682, 2010.
Do, et al., J. Vet. Sci. 13:339-344, 2012.
Kagami, et al., Oral Dis. 14(1):15-24, 2008.
King, Br. J. Pharmacol. 166:877-894, 2012.
Lai, et al. Stem Cells Dev. 19:1095-1107, 2010.
Leal-Egana & Scheibel, Biotechnol Appl Biochem. 55(3):155-67, 2010.
Maria, et al., Tissue Eng Part A. 17(9-10):1229-38, 2011.
Matsumoto, DMJ. 35:199-206, 2011.
Nagaoka, et al., Ann Biomed Eng. 38(3):683-93, 2010.
Naujok, et al., J. Tissue Eng. Regen. Med. Jan. 8, 2015. doi:10.1002/term.1857. [Epub ahead of print].
Nazarov, et al., Biomacromolecules. 5:718-726, 2004.
Nyitray, et al. Tissue Eng. Part A. Feb. 24, 2015. [Epub ahead of print].
Ryan, et al., Diabetes. 54:2060-2069, 2005.
Scharp, et al. Adv. Drug Deli. Rev. 68:35-73, 2014.
Sofia, et al., J. Biomed. Mater. Res. 54:139-148, 2001.
Wang, et al., Macromolecules. 37:6856-6864, 2004.
PCT/US2015/014994

1. A cell culture system comprising a silk fibroid scaffold, culture media, and pancreatic cells.

2. The cell culture system of claim 1, wherein the silk fibroid scaffold is coated with fibronectin.

3. The cell culture system of claim 1, wherein the pancreatic cells comprise pancreatic islet cells.

4.-7. (canceled)

8. The cell culture system of claim 1, wherein the pancreatic cells are arranged in three-dimensional cellular aggregates.

9.-15. (canceled)

16. The cell culture system of claim 1, wherein the pancreatic cells are capable of secreting insulin and/or amylin.

17.-21. (canceled)

22. The cell culture system of claim 1, wherein the culture medium comprises an insulin secretion agonist.

23. (canceled)

24. The cell culture system of claim 1, wherein the culture medium comprises insulin secreted from the pancreatic cells.

25. The cell culture system of claim 1, wherein the cell culture system comprises a three-dimensional extracellular matrix.

26. (canceled)

27. The cell culture system of claim 25, wherein the three-dimensional extracellular matrix is an extracellular matrix of bone marrow cells synthesized using a three-dimensional silk fibroin scaffold.

28. (canceled)

29. The cell culture system of claim 1, wherein the pancreatic cells are capable of constructing a three-dimensional extracellular matrix.

30.-31. (canceled)

32. The cell culture system of claim 29, wherein the three-dimensional extracellular matrix comprises collagen type IV.

33.-34. (canceled)

35. The cell culture system of claim 25, further comprising wherein the three-dimensional extracellular matrix is made from cells from the same subject wherein the pancreatic cells were obtained.

36. A method of forming a pancreatic cell-specific extracellular matrix comprising exposing the cell culture system of claim 29 to ascorbic acid.

37.-39. (canceled)

40. The method of claim 36, further comprising decellularizing the extracellular matrix.

41.-46. (canceled)

47. A method of producing pancreatic cells capable of treating a pancreatic condition, the method comprising incubating pancreatic cells and/or precursors of pancreatic cells with a three-dimensional extracellular matrix.

48. The method of producing pancreatic cells of claim 47, wherein the three-dimensional extracellular matrix is an extracellular matrix generated by bone marrow cells.

49. (canceled)

50. The method of producing pancreatic cells of claim 47, wherein the three-dimensional extracellular matrix is an extracellular matrix synthesized using a three-dimensional silk fibroin scaffold.

51. (canceled)

52. The method of claim 47, wherein the precursors of pancreatic cells are pluripotent stem cells.

53. The method of claim 52, wherein the pluripotent stem cells are mesenchymal stem cells.

54. The method of claim 47, further comprising treating a pancreatic condition in a subject comprising providing to the subject the pancreatic cells produced.

55.-73. (canceled)
